(19) United States
(12) Patent Application Publication
(10) Pub. No.: US 2009/0274265 A1
(43) Pub. Date: Nov. 5, 2009

(54) CONTINUOUS COMPUTER TOMOGRAPHY PERFORMING SUPER-SHORT-SCANS AND STRONGER WEIGHTING OF MOST RECENT DATA

(75) Inventors: Thomas Koehler, Norderstedt (DE); Peter Forthmann, Hamburg (DE); Michael Grass, Buchholz in der Nordheide (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 11/913,818

(22) PCT Filed: May 3, 2006
(86) PCT No.: PCT/IB06/51382

§ 371 Date: Nov. 7, 2007

(30) Foreign Application Priority Data
May 12, 2005 (EP) 05103983.2

(51) Int. Cl. A61B 6/00; H05G 1/60

(52) U.S. Cl. 378/15; 378/19

(57) ABSTRACT

A computer tomography apparatus and method, a computer-readable medium and a program element are provided for examining a region of interest (ROI) of an object or patient in real-time. When only a region of interest is to be reconstructed, it is sufficient to rotate the radiation source and detector elements such that they cover a circular arc whose extension is less than π+α, α being the beam angle of the radiation source. This scanning range is called super-short-scan. Super-short-scans produce less data. Consequently image reconstruction is quicker which is very preferable for real-time CT. The CT data can furthermore be weighted in a manner that data detected at the end of a super-short-scan are weighted stronger than data detected at the beginning of a super-short-scan.

The invention relates to the field of X-ray imaging. In particular, the invention relates to a computer tomography apparatus, to a method of examining an object of interest with a computer tomography apparatus, to a computer-readable medium and to a program element.

Computed tomography (CT) is a process of using digital processing to generate a three-dimensional image of the internals of an object from a series of two-dimensional X-ray images taken around a single axis of rotation. The reconstruction of CT images can be done by applying appropriate algorithms.

Noo, F, Defrise, M, Clackdoyle, R, Kudo, H, 2002, “Image reconstruction from fan-beam projections on less than a short scan”, Phys. Med. Biol. 47, 2525-2546 discloses two-dimensional image reconstruction from fan-beam projections implementing a filtered-backprojection algorithm derived for reconstruction of images according to data acquired using a computed tomography (CT) apparatus.

Kudo, H, Noo, F, Defrise, M and Rodet, T, 2003 “New approximate filtered backprojection algorithm for cone-beam helical CT with redundant data”, In: Nuclear Science Symposium Conference Record, IEEE discloses a filtered backprojection algorithm for cone-beam helical computed tomography.

CT fluoroscopy is a process of using CT in a continuous imaging mode particularly to assist in biopsies and other image guided procedures. However, in known CT fluoroscopy systems, which may also be denoted as continuous CT systems (CCT), displaying determined images of an object of interest in real-time is difficult, since the huge amount of data and the complexity of the reconstruction algorithms require quite a long time to reconstruct the images from the acquired data. Consequently, latency is one of the most important issues in CCT.

There may be a need for a computer tomography apparatus, which has a sufficiently low effective latency when determining an image from acquired data.

According to the invention, a computer tomography apparatus, a method of examining an object of interest with a computer tomography apparatus, a computer-readable medium and a program element with the features according to the independent claims are provided.

According to the invention, a computer tomography apparatus for examination of an object of interest is provided, comprising an electromagnetic radiation source adapted to rotate around the object of interest and adapted to emit an electromagnetic radiation beam having a predetermined beam angle to the object of interest. Further, the computer tomography apparatus may comprise detecting elements adapted to rotate around the object of interest and adapted to repeatedly detect scan segments of electromagnetic radiation emitted by the electromagnetic radiation source and passed through the object of interest, wherein the scan segments have an angle which is smaller than a sum of 180° and a beam angle which would be required for covering the entire object of interest. The computer tomography apparatus may further comprise a determination unit adapted to repeatedly determine images of the object of interest based on an analysis of the detected scan segments.

According to the invention, a method of examining an object of interest with a computer tomography apparatus is further provided, wherein the method comprising the steps of rotating an electromagnetic radiation source and detecting elements around the object of interest, emitting, by means of the electromagnetic radiation source, an electromagnetic radiation beam having a predetermined beam angle to the object of interest, and repeatedly detecting, by means of the detecting elements, scan segments of electromagnetic radiation emitted by the electromagnetic radiation source and passed through the object of interest. The scan segments may have an angle which is smaller than a sum of 180° and a beam angle which would be required for covering the entire object of interest. Moreover, images of the object of interest may be repeatedly determined based on an analysis of the detected scan segments.

According to the invention, a computer-readable medium is provided, in which a computer program of examining an object of interest with a computer tomography apparatus is stored which, when being executed by a processor, is adapted to control or carry out the above-mentioned method steps.

Furthermore, according to the invention, a program element of examining an object of interest is provided, which, when being executed by a processor, is adapted to control or carry out the above-mentioned method steps.

The examination of an object of interest according to the invention can be realized by a computer program, i.e. by software, or by using one or more special electronic optimization circuits, i.e. in hardware, or in hybrid form, i.e. by means of software components and hardware components. The computer-readable medium and the program element may be implemented in a control system for controlling a computer tomography apparatus.

Exemplary embodiments of the invention are disclosed in the dependent claims.

According to an exemplary embodiment of the invention, a computer tomography apparatus for examination of an object of interest is provided, comprising an electromagnetic radiation source adapted to rotate around the object of interest and adapted to emit an electromagnetic radiation beam to the object of interest, and detecting elements adapted to rotate around the object of interest and adapted to repeatedly detect scan segments of electromagnetic radiation emitted by the electromagnetic radiation source and passed through the object of interest. The computer tomography apparatus may further comprise a determination unit adapted to repeatedly determine images of the object of interest based on an analysis of the detected scan segments so that the images are provided to be displayable essentially in real-time. The determination unit may further be adapted in such a manner that data related to a scan segment which data are detected at an end portion of a scan segment detection time interval are weighted stronger than data detected at a beginning portion of the scan segment detection time interval.

According to another exemplary embodiment of the invention, a method of examining an object of interest with a computer tomography apparatus is provided, the method comprising the steps of rotating an electromagnetic radiation source and detecting elements around the object of interest, emitting, by means of the electromagnetic radiation source, an electromagnetic radiation beam to the object of interest, and repeatedly detecting, by means of the detecting elements, scan segments of electromagnetic radiation emitted by the electromagnetic radiation source and passed through the object of interest. Further, images of the object of interest may be repeatedly determined based on an analysis of the detected scan segments so that the images are provided to be displayable essentially in real-time, wherein data related to a scan segment which data are detected at an end portion of a scan segment detection time interval are weighted stronger than data detected at a beginning portion of the scan segment detection time interval.

According to one aspect of the invention, a computer tomography apparatus is provided which allows to display images derived from continuously captured detection data in real-time. This is enabled by adjusting the scan segments as so-called super-short scan segments having a scan angle (scanned during the rotation of the electromagnetic radiation source and the detecting elements which may be mounted on a gantry) which is smaller than π (that is to say half a rotation) plus a beam angle (for instance a fan angle of the beam) which would cover the whole object of interest. In other words, only data related to a sub-portion of the object of interest, for instance a central circular region of interest of the object, is evaluated, so that only the highly relevant data related to this reduced portion of the object of interest are considered for an analysis, that is to say to determine or reconstruct the image of at least a part of the object of interest. According to the invention, the concept of a super-short scan which has been introduced in another context by the above cited reference of Noo et al. 2002 is implemented in the frame of a continuous CT system (or CT fluoroscopy system) to allow displaying the determined images almost in real-time. Therefore, the computer tomography fluoroscopy apparatus according to the invention allows to generate some kind of “movie” of the interesting portion of the object of interest which may, for instance, be provided to a radiologist for planning or controlling or carrying out a treatment like a biopsy. Less data to be analyzed means a shorter analysis time, and thus a reduced latency.

According to another aspect of the invention, an advantageous scheme for weighting the acquired data is provided. According to this weighting scheme, preferably and primarily those data of a scan segment are selected for a subsequent reconstruction of the image which data have been acquired only a short time ago (namely at the end of the procedure of acquiring data related to the scan segment), whereas data of the scan segment which have been acquired quite a long time ago (namely at the beginning of the procedure of acquiring data related to the scan segment) are lower prioritized.

The invention relates to different but strongly related aspects. According to one aspect, the real latency is reduced by providing a super short scan allowing a processing with a reduced amount of data, thus accelerating the analysis or reconstruction. According to another aspect, the effective latency is reduced by predominantly using data for the analysis which data have been acquired recently, namely at the end of a scan. Both measures, taken isolated or in combination, allow to reduce latency in the frame of a CT fluoroscopy system.

A scenario may occur, in which a radiologist may desire to take a sample of tissue of a lung of a patient. For this purpose, the radiologist may have to insert a needle in the lung. In order to assist the radiologist in this dangerous procedure, it is advantageous to provide the radiologist with a time-resolved image of the organ (e.g. the lung) which is to be treated by the radiologist. This is enabled by the invention by providing a significantly simplified and accelerated reconstruction algorithm which includes a reduced amount of data to be processed, namely only the data related to a region of interest within the object of interest. Further, by predominantly evaluating very recent data, the reliability of the image is increased. In other words, a super-short scan can implemented in the frame of a CCT apparatus, and/or an improved weighting scheme can be realized.

Thus, a real-time display of a CT image is made possible by continuously capturing data and reconstructing images with a CT scanner from a scaled down portion of interest and/or with a high degree of up-to-dateness. Thus, a very short latency is achievable, and a radiologists can be provided, for instance for the purpose of a biopsy, with highly reliable data of the object or region of interest. In other words, the invention provides a real-time CT or CT fluoroscopy apparatus which allows fast scan times and rapid image reconstruction. The real-time images may be used to guide interventional procedures such as lesion, biopsy and drainage. The images may be reconstructed with a particular frame rate, for instance 12 frames per second. Then, the real-time reconstructed data are providable to a monitor for viewing the CT fluoroscopy output.

In continuous CT (CCT), also known as CT fluoroscopy, X-ray projections of the patient are continuously acquired while the gantry rotates. A series of images/volume is reconstructed, wherein the most recent image/volume is supposed to represent the current state of the patient in order to allow an online guidance, for instance of a biopsy. According to the invention, latency being one of the most important issues in CCT is significantly reduced. In contrast to the prior art, where reconstruction is done based on data acquired along a so-called short scan segment and regardless to the timeliness of the data, the reconstruction according to the invention may implement a so-called super-short scan segment and/or may focus on recent data.

The above-mentioned reference Noo et al. 2002 which is incorporated within the disclosure of the present patent application and which discloses an algorithm which can be used to analyze data in the frame of the system according to the invention, discloses a 2D reconstruction algorithm that needs even less than a short scan segment for the reconstruction of a region of interest. By applying this algorithm, according to the invention, to the CCT technology, the latency can be significantly reduced. Another advantage of the algorithm according to Noo et al. 2002 when being applied to a CT fluoroscopy system, is that weighting the data may be performed after filtering the data. Thus, according to the invention, a sliding window reconstruction can be performed more efficiently than in the traditional method with Parker weighting or parallel beam reconstruction after rebinning.

According to one aspect of the invention, a super-short scan may be implemented in CT fluoroscopy. According to CT fluoroscopy, constantly updated images produced by continuous rotation of a CT tube may be displayed. Thus, a real-time analysis of CT data is carried out according to the invention by using a scan angle which is less than π plus a fan angle covering the entire object of interest.

Referring to the dependent claims, further exemplary embodiments of the invention will be described.

Next, exemplary embodiments of the computer tomography apparatuses for examination of an object of interest will be described. These embodiments may also be applied for the method of examining an object of interest with one of the computer tomography apparatuses, for the computer-readable medium and for the program element.

The computer tomography apparatus may be adapted in such a manner that the determination unit determines images of only a portion of the object of interest. By taking this measure, the amount of data to be analyzed is reduced, since only data related to a part of the object of interest (for instance only an organ or only a part of an organ of a patient) are used. Particularly, the portion of the object of interest analyzed may be a central portion of the object of interest. Such a central portion may be a central circular portion of the object of interest. The portion of the object of interest should have a convex geometry, for instance may be a sphere.

The determining unit may be adapted to repeatedly determine images of the structure of the object of interest based on a sliding window reconstruction analysis of the detected scan segments. In other words, data related to different segments on, for instance, a circular trajectory on which the X-ray tube in the detector rotates, may be used for reconstructing the image of the object of interest or a part thereof.

The computer tomography apparatus may further comprise a display for displaying the determined images of the structure of the object of interest in real-time. For instance, a monitor may be provided for a radiologist to allow the radiologist to monitor the time dependence of the structure of the object of interest, for instance to plan or carry out a biopsy. Such a display can be, for instance, a cathode ray tube (CRT), a liquid crystal display (LCD) or a plasma display device.

A control unit may be provided adapted to control a treatment of the object of interest based on the images of the structure of the object of interest displayable in real-time. Particularly, the control unit may be adapted to control a biopsy of the object of interest based on the images of the object of interest displayable in real-time. This allows a user to continuously monitor the recent structure of the object under treatment which allows a more reliable and less dangerous treatment of the object of interest.

The determining unit may be adapted to repeatedly determine images of the structure of the object of interest based on an analysis which includes filtering detected data related to the scan segments and subsequently weighting the filtered data related to the scan segments. In other words, according to the invention, weighting may be applied after filtering. This feature may allow to reduce the computational costs for a reconstruction, particularly for a sliding window reconstruction, so that the latency characteristics may be further improved.

The determination unit may further be adapted to weight data related to a scan segment using a discontinuous weighting function. In other words, a non-smooth weighting function (like a step function) may be implemented in the case of the invention which allows to reconstruct the images with less computational burden, and thus in a fast manner. It may be advantageous to select the weighting function in such a manner that artifacts are suppressed.

The determination unit may be adapted in such a manner that data related to a scan segment which data are detected at an end portion of a scan segment detection time interval are weighted stronger than data detected at a beginning portion of the scan segment detection time interval. Within a scan segment, for instance a super-short scan segment, an angle of more than π may be covered by the X-ray tube and the detector. The data captured at the end of this angle range are more recent than the data captured at the beginning. According to the weighting scheme of the invention, predominantly those data may be used for an analysis which have been captured quite recently so that the image reconstructed and displayed relates to a geometry of the object of interest at a time which is not long ago. In other words, the weighting function may be selected in such a manner that the “young” data of a scan segment are used for the analysis, wherein relatively “old” data are omitted or used in a less intense manner.

The determination unit may further be adapted to repeatedly determine three-dimensional images of the structure of the object of interest. Such steric or three-dimensional images can be calculated from two-dimensional projections.

Particularly, the computer tomography apparatus according to the invention may be adapted as a computer tomography fluoroscopy apparatus or a continuous computed tomography apparatus. In the frame of this technology, the provision of real-time images of an object under investigation is particularly advantageous.

The computer tomography apparatus according to the invention may be adapted in a manner that the electromagnetic radiation source and the detection elements may rotate around the object of interest along a circular trajectory. In other words, a circular scan may be carried out, that is the electromagnetic radiation source and the detection elements may be arranged on a gantry to rotate around the object under investigation. A circular scan may be particularly advantageous when a multi-slice detector is used. However, also a single-slice detector may be used.

The computer tomography apparatus may comprise a collimator arranged between the electromagnetic radiation source and the detecting elements, wherein the collimator may be adapted to collimate an electromagnetic radiation beam emitted by the electromagnetic radiation source to form a fan-beam or a cone-beam with the predetermined beam angle. Such a collimator thus allows to define the radiation profile. The invention is primarily directed to a fan-beam geometry, but however may also be applied to a cone-beam geometry.

The detecting elements of the computer tomography apparatus may form a single-slice detector array. This configuration allows to construct a computer tomography apparatus with low effort.

Alternatively, the detecting elements may form a multi-slice detector. This configuration can be advantageous particularly when combined with a circular scan.

The computer tomography apparatus may be configured as one of the group consisting of a medical application apparatus, a material testing apparatus and a material science analysis apparatus. The invention creates a high-quality automatic system that can automatically recognize certain types of material in a time-resolved manner. Such a system may have employed the computer tomography apparatus of the invention with an X-ray radiation source for emitting X-rays which are transmitted through or passed through the examined object or person to a detector allowing to detect a region of interest within the object of interest in a high accuracy manner.

The aspects defined above and further aspects of the invention are apparent from the examples of embodiment to be described hereinafter and are explained with reference to these examples of embodiment.

The invention will be described in more detail hereinafter with reference to examples of embodiment but to which the invention is not limited.

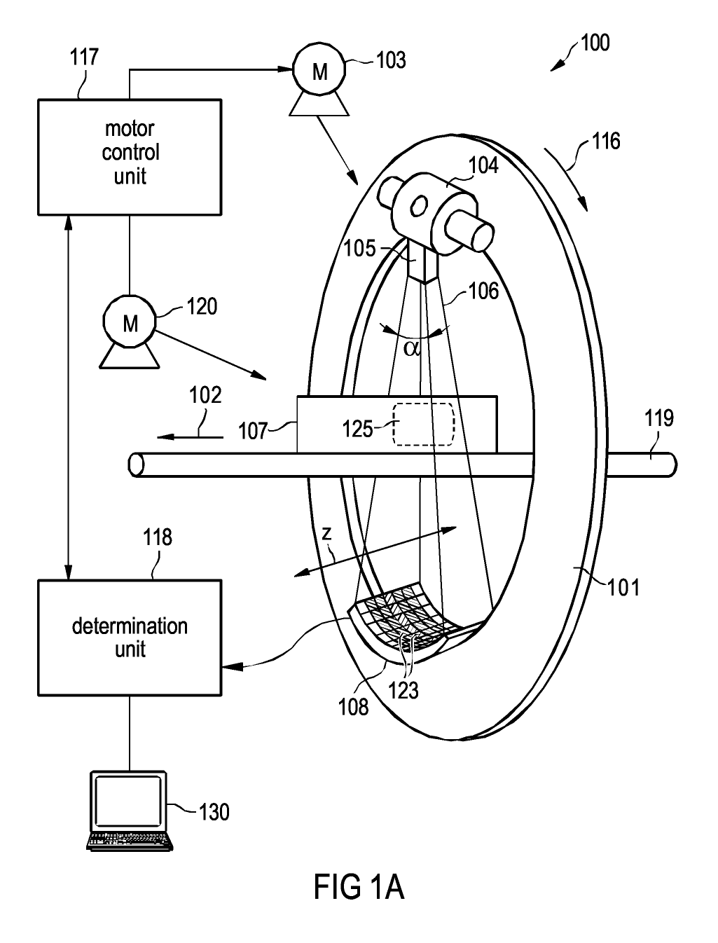

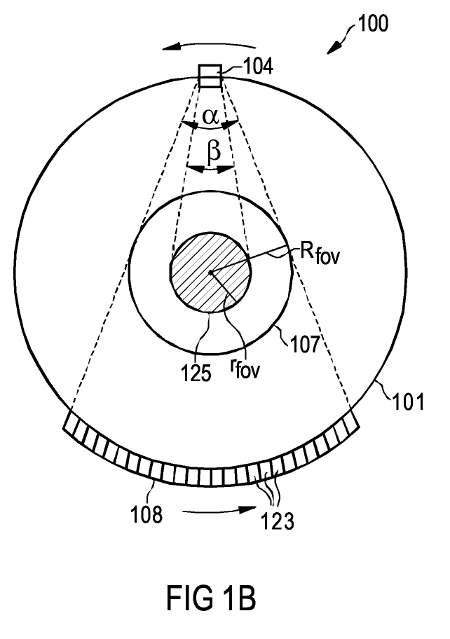

FIG. 1A shows a computer tomography apparatus according to an exemplary embodiment of the invention.

FIG. 1B shows a schematic view of the geometry of a super-short scan performed with the computer tomography apparatus of FIG. 1A.

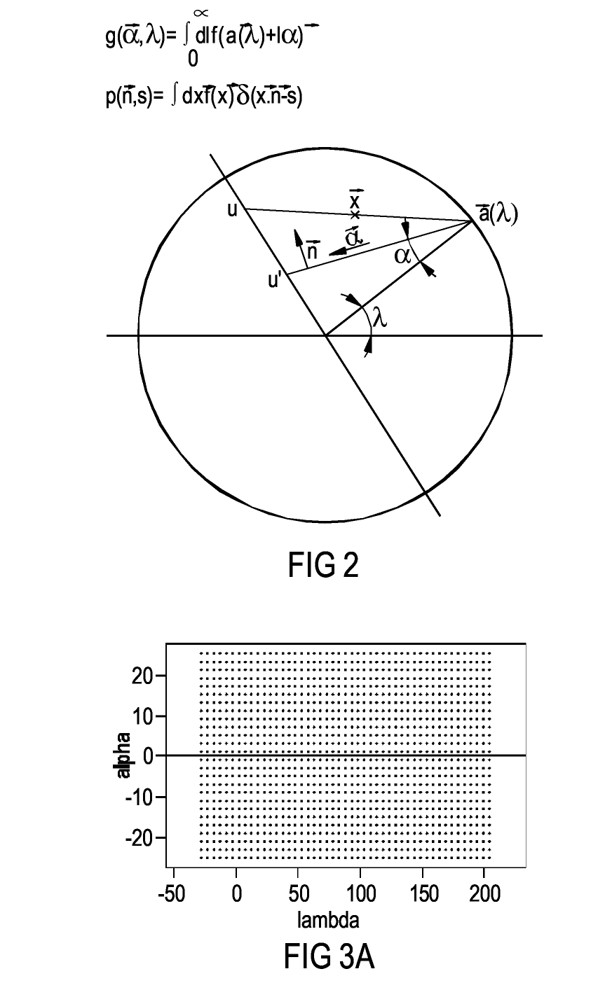

FIG. 2 illustrates a ray geometry according to an exemplary embodiment of the invention.

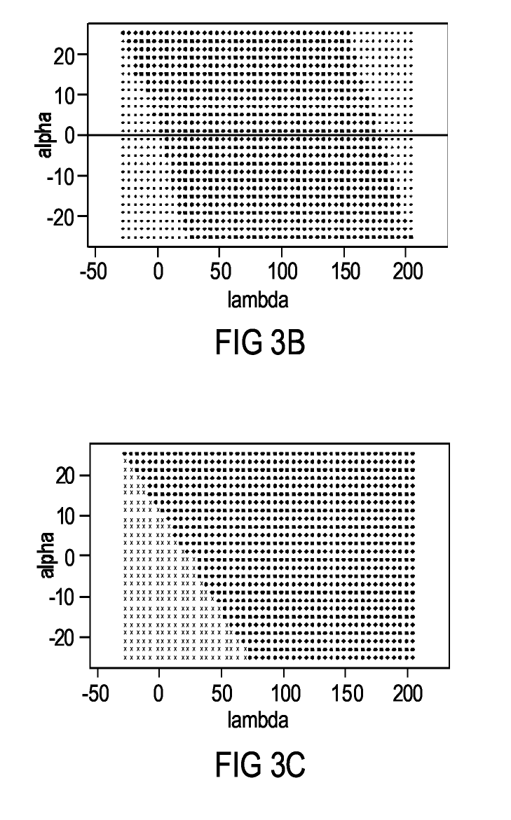

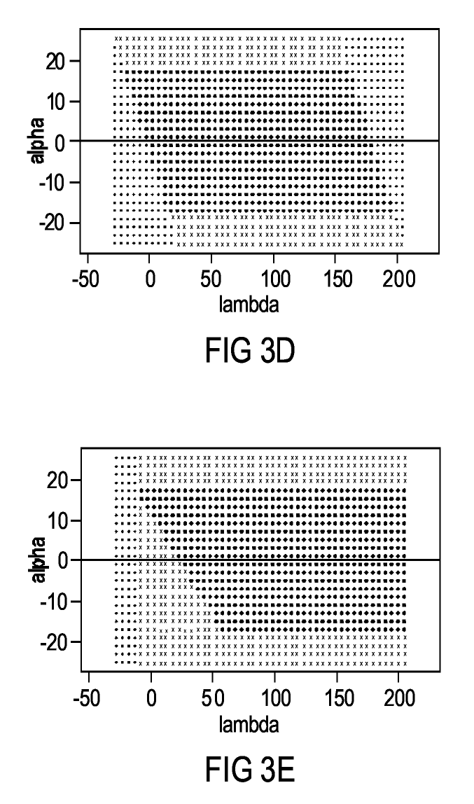

FIG. 3A shows data acquired during a method of examining an object of interest with a computer tomography apparatus,

FIG. 3B shows, for conventional parallel rebinning, data acquired during a scan (dots) and data used for pre-processing and back-projection (bold dots),

FIG. 3C shows, for a method of examining an object of interest with a computer tomography apparatus according to an exemplary embodiment of the invention, data used for pre-processing but not for back-projection (crosses) and data used for pre-processing and back-projection (bold dots),

FIG. 3D shows, for conventional parallel rebinning with a reconstruction for a reduced field of view (fov), data acquired during a scan (dots), data used for pre-processing but not for back-projection (crosses) and data used for pre-processing and back-projection (bold dots),

FIG. 3E shows, for a method of examining an object of interest with a computer tomography apparatus according to an exemplary embodiment of the invention with a reconstruction for a reduced field of view (fov), data acquired during a scan (dots), data used for pre-processing but not for back-projection (crosses) and data used for a pre-processing and back-projection (bold dots).

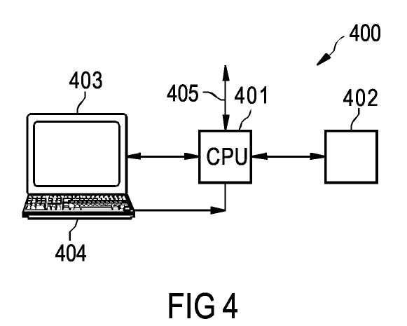

FIG. 4 shows an exemplary embodiment of a data processing device to be implemented in a computer tomography apparatus of the invention.

The illustration in the drawings is schematically. In different drawings, similar or identical elements are provided with the same reference signs.

FIG. 1A shows an exemplary embodiment of a computed tomography scanner system according to the present invention.

With reference to this exemplary embodiment, the present invention will be described for the application in examination of an organ of a human patient. However, it should be noted that the present invention is not limited to this application, but may also be applied in other fields of medical imaging, or other industrial applications such as material testing.

The computer tomography apparatus 100 depicted in FIG. 1A is a fan-beam CT scanner. However, the invention may also be carried out with a cone-beam geometry. The CT scanner depicted in FIG. 1A comprises a gantry 101, which is rotatable around a rotational axis 102. The gantry 101 is driven by means of a motor 103. Reference numeral 104 designates a source of radiation such as an X-ray source, which, according to an aspect of the present invention, emits polychromatic or essentially monochromatic radiation.

Reference numeral 105 designates an aperture system which forms the radiation beam emitted from the radiation source to a fan-shaped radiation beam 106. The fan-beam 106 is directed such that it penetrates an object of interest 107 arranged in the center of the gantry 101, i.e. in an examination region of the CT scanner, and impinges onto the detector 108. As may be taken from FIG. 1A, the detector 108 is arranged on the gantry 101 opposite to the source of radiation 104, such that the surface of the detector 108 is covered by the fan-beam 106. The detector 108 depicted in FIG. 1A comprises a plurality of detector elements 123 each capable of detecting X-rays which have passed through the object of interest 107.

During scanning the object of interest 107, the source of radiation 104, the aperture system 105 and the detector 108 are rotated along the gantry 101 in the direction indicated by an arrow 116. For rotation of the gantry 101 with the source of radiation 104, the aperture system 105 and the detector 108, the motor 103 is connected to a motor control unit 117, which is connected to a determination unit 118 (which might also be denoted as a calculation unit).

In FIG. 1A, the object of interest 107 is a human patient which is disposed on a mounting table 119. During the scan of the object of interest 107, the gantry 101 rotates around the human patient 107. The mounting table 119 may displace the object of interest 107 along a direction parallel to the rotational axis 102 of the gantry 101. The object of interest 107 may be scanned along a circular scan path.

Further, it shall be emphasized that, as an alternative to the fan-beam configuration shown in FIG. 1A, the invention can be realized by a cone-beam configuration. In order to generate a primary fan-beam, the aperture system 105 may be configured as a slit collimator.

The detector 108 is connected to a determination unit 118. The determination unit 118 receives the detection result, i.e. the read-outs from the detector elements 123 of the detector 108 and determines a scanning result on the basis of these read-outs. Furthermore, the determination unit 118 communicates with the motor control unit 117 in order to coordinate the movement of the gantry 101 with motor 103 and may communicate with the X-ray source 104 to control radiation dose and exposure time.

The determination unit 118 may be adapted for reconstructing an image from read-outs of the detector 108. A reconstructed image generated by the control unit 118 may be output by a display 130 which may also include means for user-interaction, for instance a keypad, a computer mouse, etc.

The determination unit 118 may be realized by a data processor to process read-outs from the detector elements 123 of the detector 108.

The computer tomography apparatus 100 comprises the X-ray source 104 which is adapted to emit X-rays to the object of interest 107. The collimator 105 provided between the electromagnetic radiation source 104 and the detecting elements 123 is adapted to collimate an electromagnetic radiation beam emitted from the electromagnetic radiation source 104 to form a fan-beam. The detecting elements 123 form a multi-slice detector array 108. The computer tomography apparatus 100 is configured as a medical examination apparatus.

The computer tomography apparatus 100 for examination of a patient 107 comprises the X-ray tube 104 which is adapted to rotate, mounted on the gantry 101, around the patient 107 and is adapted to emit an X-ray beam having a predetermined beam angle α to the patient 107. Further, the detecting elements 123 may rotate, mounted on the gantry 101, around the patient 107 and repeatedly detect scan segments of electromagnetic radiation emitted by the X-ray tube 104 and passed through the patient 107. The scan segments captured by the detection elements 123 have an angle which is smaller than a sum of 180° and a beam angle which would be necessary to cover the entire patient 107.

The determination unit 118 repeatedly determines images of the structure of the patient 107 based on an analysis of the detected scan segments so that the images are provided to be displayable in real-time on the display device 130. Particularly, the determination unit 118 is adapted in such a manner that only detection data related to a portion of interest 125 (for instance a lung as an organ under investigation or a circular portion within the patient 107) of the patient 107 is considered for image reconstruction. Thus, a reduced amount of data has to be processed by the determination unit 118 to determine the three-dimensional image of the portion of interest 125 to be continuously displayed on the display 107. With a sliding window reconstruction analysis of the detected scan segments, the determination unit 118 can determine the three-dimensional images of the portion of interest 125.

A radiologist, for planning or simultaneously carrying out a biopsy of the patient 107, can continuously monitor the recent image of the portion of interest 125 on the display device 130 which allows the radiologist to perform the biopsy with high accuracy and reduced risk for the health of the patient 107. The computer tomography apparatus 100 is adapted as computer tomography fluoroscopy apparatus or a continuous computer tomography apparatus.

When repeatedly determining the images of the structure of the patient 107, the determination unit 118 carries out an analysis which includes filtering data related to the detected scan segments and subsequently weighting the filtered data related to the detected scan segments. By performing weighting after filtering, the computational burden for reconstructing the images, and thus the real-time functionality of the system, is improved.

As will be described in the following, the determination unit weights the data related to a scan segment using a discontinuous weighting function, namely a kind of step function. Particularly, data related to a scan segment which data are detected at an end portion of a scan segment detection time interval are weighted stronger than data detected at the beginning portion of the scan segment detection time interval. Thus, the image displayed on the display 130 is a very recent illustration of the portion of interest 125.

In the following, referring to FIG. 1B, a schematic view of portions of the computer tomography apparatus 100 of FIG. 1A are shown to illustrate the geometry of the apparatus.

As can be seen in FIG. 1B, the X-ray tube 104 and the detector 108 rotate on the gantry 101. During the rotation, the X-ray source 104 emits electromagnetic radiation within a segment of an angle α which covers essentially the entire diameter of the patient 107. However, for a subsequent analysis and reconstruction of the image, only a part of the captured data needs to be used, for instance in a case that a reduced field of view is sufficient (for instance when only an image of a reduced portion 125 of the object 107 shall be determined which relates to an angle β). The restriction to a circular central portion 125 of the patient 107 reduces the angular range over which the X-ray tube 104 and the detector 108 have to rotate along the gantry 101 to a super-short.

In the following, a method of reconstructing the images and of carrying out the measurement according to an exemplary embodiment of the invention will be described.

According to this exemplary embodiment, a method use for the CCT apparatus 100 is a super-short scan algorithm similar to that of the above-mentioned references Noo et al. 2002 and Kudo et al. 2003 providing a 2D-method, but it is already generalized to 3D in a usual way.

The geometry of this reconstruction scheme is shown in FIG. 2.

Let w(\vec{n}, λ) be a weighting function that acts on the projection values of measured rays so that redundant rays are weighted according to their multiplicity

$$\begin{matrix}{\sum }_{i}^{N ( \vec{n} , s )} w ( {λ}_{i} , \vec{n} ) = 1 & ( 1 ) \\ s = \vec{n} \cdot \vec{a} ( {λ}_{i} ) & ( 2 )\end{matrix}$$

By using the relationship between the ramp and the Hilbert filter

$${h}_{R} ( s ) * p ( \vec{n} , s ) ∘ - • \vert v \vert P ( \vec{n} , v ) = \frac{1}{2 π} ( - i sgn ( v ) ) ( i 2 π v ) P ( \vec{n} , v ) ∘ - • \frac{1}{2 π} {h}_{H} ( s ) * \frac{\partial }{\partial s} p ( \vec{n} , s ) \qquad ( 3 )$$

and the so-called Hamaker relation (see Hamaker, C et al. “The divergent beam x-ray transform”, Rocky Mountain Journal of Mathematics, 6:253-283, 1980):

p_{H}(\vec{n},s)|_{s=\vec{a}(λ)·\vec{n}}=g_{H}(\vec{n},λ) (4)

From this, the following exact reconstruction algorithm can be derived (see Noo et al 2002, particularly equations (26) and (38)):

$$\begin{matrix}f ( \vec{x} ) = \int d λ \frac{1}{R - x cos λ - y sin λ} w ( u , λ ) {g}^{F } ( u , λ ) & ( 5 ) \\ {g}^{F} ( u , λ ) = \frac{1}{2 π} \int d {u}^{′} {h}_{H} ( u - {u}^{′} ) \frac{R}{\sqrt{{R}^{2} + {u}^{′2}}} ( \frac{\partial }{\partial λ} + \frac{\partial {u}^{′}}{\partial λ} \frac{\partial }{\partial {u}^{′}} ) g ( {u}^{′} , λ ) & ( 6 )\end{matrix}$$

In equations (5), (6), g^{F} is a filter function, and w is a weighting function.

In contrast to standard fan-beam reconstruction, according to the reconstruction scheme of the present embodiment of the invention, weighting is applied after filtering. This implies that there is no need (as in Parker weighting) to use a smooth weighting function in order to avoid artifacts.

This recognition will be exploited in the following.

Applying the algorithm of Noo et al. 2002 to CCT, it is advantageous to recognize that for CCT applications, the requirements on spatial resolution are not very demanding. Thus, it is sufficient to approximate the derivative with respect to λ by a subtraction of subsequent projections in order to achieve a negligible additional latency for this step on the pre-processing.

Assuming that a region of interest (ROI) fits completely into a centred circular region with radius r_{fov}, which is typically smaller than the scan fields of view (fov) of the system with radius R_{fov}.

The short scan segment bounded by λ₁ and λ₂ with λ₂>λ₁ has a length:

λ₂−λ₁=π+2 arcsin(R_{fov}/R) (7)

while a super-short scan segment has a length

λ₂−λ₁=π+2 arcsin(r_{fov}/R) (8)

For the reconstruction of the object inside the region of interest (ROI) using the algorithm of Noo et al. 2002, only a super-short scan segment is required.

For CCT, it is an aim to use the most recent data as strong as possible. This may be achieved in the framework of the algorithm of Noo et al. 2002 by using the weighting function:

$$\begin{matrix}w ( u , λ ) = \{ \begin{matrix}1 & for α ( u ) \geq ( {λ}_{2} - λ - π ) / 2 \\ 0 & else\end{matrix} \\ where & ( 9 ) \\ α ( u ) = arctan ( u / R ) & ( 10 )\end{matrix}$$

α(u) is the fan angle of the ray that hits the detector at u.

It the following, referring to FIG. 3A to FIG. 3E, it will be described how latency may be reduced with the schemes according to the invention.

The diagrams shown in FIG. 3A to FIG. 3E plot, along the abscissa, the source angle λ, and, along the ordinate, the fan-angle α.

One might say that the source angle λ plotted along the abscissa of the diagrams of FIG. 3A to FIG. 3E relate to a time axis of measuring. Data on the right hand side of the abscissa of the diagrams of FIG. 3A to FIG. 3E are taken at the end of a scan, and data on the left hand side of the abscissa of the diagrams of FIG. 3A to FIG. 3E are taken at the beginning of a scan.

FIG. 3A shows, as dots, data acquired during a method of examining an object of interest with a computer tomography apparatus.

FIG. 3B shows, for a conventional parallel rebinning image reconstruction method, data acquired during a scan (dots) and data used for pre-processing and back-projection (bold dots). The small dots indicate measured data which are not used at all. The bold dots relate to data used for pre-processing and back-projection. However, many very recent data are not used (see triangle of non-bold dots at the right hand side of FIG. 3B). Thus, the latency is quite large in the case of the conventional parallel rebinning image reconstruction method.

FIG. 3C shows, for an image reconstruction method according to an exemplary embodiment of the invention, data used for pre-processing but not for back-projection (crosses) and data used for pre-processing and back-projection (bold dots). As can be seen from FIG. 3C, predominantly very recent data are used for reconstruction, which results in a reduces effective latency.

Comparing FIG. 3B with FIG. 3C, both methods use the same range of projections, but the method according to FIG. 3C uses on average more recent data, thus the effective latency is smaller. FIG. 3B and FIG. 3C relate to a situation in which no reduced field of view is investigated, but the entire field of view (r_{fov}=R_{fov}).

FIG. 3D and FIG. 3E relate to a situation in which a reduced field of view is investigated, that is to say r_{fov}<R_{fov}. In the following, data usage for image reconstruction will be described for FIG. 3D and FIG. 3E.

FIG. 3D shows, for conventional parallel rebinning with a reconstruction for a reduced field of view (fov), data acquired during a scan (dots), data used for pre-processing but not for back-projection (crosses) and data used for pre-processing and back-projection (bold dots). However, many very recent data are not used (see triangle of non-bold dots at the right hand side of FIG. 3D). Thus, the latency is quite large in the case of the conventional parallel rebinning image reconstruction method.

FIG. 3E shows, for a method of examining an object of interest with a computer tomography apparatus according to an exemplary embodiment of the invention with a reconstruction for a reduced field of view (fov), data acquired during a scan (dots), data used for pre-processing but not for back-projection (crosses) and data used for a pre-processing and back-projection (bold dots). As can be seen from FIG. 3E, predominantly very recent data are used for reconstruction, which results in a reduces effective latency. Furthermore, the four left columns of data (i.e. very old data) are not needed for the reconstruction at all, so that less data have to be processed which results in a reduced processing time. Therefore, both effective and real latency are significantly reduced in the case of FIG. 3E.

Concluding, the parallel-rebinning method according to FIG. 3D uses still all projections, while the method according to FIG. 3E does not need the last four projections. Thus, latency is further reduced.

As can be seen in FIG. 3C, FIG. 3E, the very recent data on the right hand side, that is to say at high source angles λ, are used for the reconstruction in a stronger manner than in case of FIG. 3B, FIG. 3D so that the image received is a very recent image of the object of interest.

For a full fov reconstruction, the same range of projection angles is required as for the traditional method and the new method. However, the mean age of the used data is less for the new method, resulting in a smaller effective latency. For a smaller ROI, less fan-beam projections are required using the method according to the invention, resulting in a further reduced real latency.

It should be noted that the weighting is constant over a rather large range of source angles λ. This implies that a partial backprojection of constantly weighted projections can be shared among subsequent images to reduce the overall computational costs.

Specifically, according to an exemplary embodiment of the invention, reconstruction is performed with the formula of equations (5) and (6).

In these equations, h_{H} denotes the convolution kernel of the Hilbert transform. Two main features of this algorithm according to the invention are that it facilitates a reconstruction using data of less than a short scan and that weighting is applied after filtering. The first feature can be used to reduce the latency in CCT compared to other construction techniques and the second feature reduces the computational costs for sliding window reconstruction, which is mandatory in CCT. For a continuous reconstruction of a region of interest that fits completely in a centred circular region of radius r_{fov}, a super-short scan segment is bounded by projection angles according to equation (8). According to the described invention, a weighting function according to equation (9) may be used that results in the minimum possible latency.

FIG. 4 depicts an exemplary embodiment of a data processing device 400 according to the present invention for executing an exemplary embodiment of a method in accordance with the present invention. The data processing device 400 depicted in FIG. 4 comprises a central processing unit (CPU) or image processor 401 connected to a memory 402 for storing an image depicting an object of interest, such as a patient or an item of baggage. The data processor 401 may be connected to a plurality of input/output network or diagnosis devices, such as an MR device or a CT device. The data processor 401 may furthermore be connected to a display device 403, for example a computer monitor, for displaying information or an image computed or adapted in the data processor 401. An operator or user may interact with the data processor 401 via a keyboard 404 and/or other output devices, which are not depicted in FIG. 4. Furthermore, via the bus system 405, it is also possible to connect the image processing and control processor 401 to, for example a motion monitor, which monitors a motion of the object of interest. In case, for example, a lung of a patient is imaged, the motion sensor may be an exhalation sensor. In case the heart is imaged, the motion sensor may be an electrocardiogram (ECG).

Exemplary technical fields, in which the present invention may be applied advantageously, include baggage inspection, medical applications, material testing, and material science. An improved image quality and a reduced amount of calculations in combination with a low effort may be achieved. Also, the invention can be applied in the field of heart scanning to detect heart diseases.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

1. A computer tomography apparatus for examining an object of interest, the computer tomography apparatus comprising:
an electromagnetic radiation source adapted to rotate around the object of interest Hand adapted to emit an electromagnetic radiation beam having a predetermined beam angle to the object of interest;
detecting elements adapted to rotate around the object of interest and adapted to repeatedly detect scan segments of electromagnetic radiation emitted by the electromagnetic radiation source and passed through the object of interests, wherein the scan segments have an angle which is smaller than a sum of 180° and a beam angle which would be required for covering the entire object of interest;
a determination unit adapted to repeatedly determine images of the object of interest based on an analysis of the detected scan segments.

2. The computer tomography apparatus according to claim 1, wherein the determination unit is adapted to determine images of only a portion of the object of interest.

3. The computer tomography apparatus according to claim 1, wherein the determination unit is adapted to determine images of only a central portion of the object of interest.

4. The computer tomography apparatus according to claim 1, wherein the determination unit is adapted to determine images of only a central circular portion of the object of interests.

5. The computer tomography apparatus according to claim 1, wherein the determination unit is adapted to determine images of only a portion of the object of interest Shaving a convex geometry.

6. The computer tomography apparatus according to claim 1, wherein the determination unit is adapted to repeatedly determine images of the object of interest based on a sliding window reconstruction analysis of the detected scan segments.

7. The computer tomography apparatus according to claim 1, comprising a display (for displaying the determined images of the object of interest essentially in real-time.

8. The computer tomography apparatus according to claim 1, comprising a control unit adapted to control a treatment of the object of interest based on the images of the object of interest displayable essentially in real-time.

9. The computer tomography apparatus according to claim 1, comprising a control unit adapted to control a biopsy of the object of interest based on the images of the object of interest displayable essentially in real-time.

10. The computer tomography apparatus according to claim 1, wherein the determination unit Dais adapted to repeatedly determine images of the object of interest based on an analysis which includes filtering data related to the detected scan segments and subsequently weighting the filtered data related to the detected scan segments.

11. The computer tomography apparatus according to claim 1, wherein the determination unit is adapted to weight data related to a scan segment using a discontinuous weighting function.

12. The computer tomography apparatus according to claim 1, wherein the determination unit is adapted in such a manner that data related to a scan segment which data are detected at an end portion of a scan segment detection time interval are weighted stronger than data detected at a beginning portion of the scan segment detection time interval.

13. The computer tomography apparatus according to claim 1, wherein the determination unit is adapted to repeatedly determine three-dimensional images of the object of interests.

14. The computer tomography apparatus according to claim 1, being adapted as a computer tomography fluoroscopy apparatus.

15. The computer tomography apparatus according to claim 1, being adapted in a manner that the electromagnetic radiation source and the detection elements rotate around the object of interest along a circular trajectory.

16. The computer tomography apparatus according to claim 1, comprising a collimator arranged between the electromagnetic radiation source and the detecting elements, the collimator being adapted to collimate an electromagnetic radiation beam emitted by the electromagnetic radiation source to form a fan-beam or a cone-beam having the predetermined beam angle.

17. The computer tomography apparatus according to claim 1, wherein the detecting elements form a single-slice detector array.

18. The computer tomography apparatus according to claim 1, wherein the detecting elements form a multi-slice detector array.

19. The computer tomography apparatus according to claim 1, configured as one of the group consisting of a medical application apparatus, a material testing apparatus and a material science analysis apparatus.

20. A method of examining an object of interest with a computer tomography apparatus, the method comprising the steps of:
rotating an electromagnetic radiation source and detecting elements around the object of interest;
emitting, by means of the electromagnetic radiation source, an electromagnetic radiation beam having a predetermined beam angle to the object of interest;
repeatedly detecting, by means of the detecting elements, scan segments of electromagnetic radiation emitted by the electromagnetic radiation source and passed through the object of interest, wherein the scan segments have an angle which is smaller than a sum of 180° and a beam angle which would be required for covering the entire object of interest;
repeatedly determining images of the object of interest based on an analysis of the detected scan segments.

21. A computer-readable medium, in which a computer program of examining an object of interest with a computer tomography apparatus is stored which, when being executed by a processors, is adapted to control the steps of:
rotating an electromagnetic radiation source and detecting elements around the object of interest;
emitting, by means of the electromagnetic radiation source, an electromagnetic radiation beam having a predetermined beam angle to the object of interest;
repeatedly detecting, by means of the detecting elements, scan segments of electromagnetic radiation emitted by the electromagnetic radiation source and passed through the object of interest, wherein the scan segments have an angle which is smaller than a sum of 180° and a beam angle which would be required for covering the entire object of interest;
repeatedly determining images of the object of interest based on an analysis of the detected scan segments.

22. A program element of examining an object of interest, which, when being executed by a processors, is adapted to control the steps of:
rotating an electromagnetic radiation source and detecting elements around the object of interest;
emitting, by means of the electromagnetic radiation source, an electromagnetic radiation beam having a predetermined beam angle to the object of interest;
repeatedly detecting, by means of the detecting elements, scan segments of electromagnetic radiation emitted by the electromagnetic radiation source and passed through the object of interest, wherein the scan segments have an angle which is smaller than a sum of 180° and a beam angle which would be required for covering the entire object of interest;
repeatedly determining images of the object of interest based on an analysis of the detected scan segments.

23. A computer tomography apparatus for examination of an object of interest, the computer tomography apparatus comprising:
an electromagnetic radiation source adapted to rotate around the object of interest and adapted to emit an electromagnetic radiation beam to the object of interest;
detecting elements adapted to rotate around the object of interest and adapted to repeatedly detect scan segments of electromagnetic radiation emitted by the electromagnetic radiation source and passed through the object of interest;
a determination unit adapted to repeatedly determine images of the object of interest based on an analysis of the detected scan segments so that the images are provided to be displayable essentially in real-time, wherein the determination unit is adapted in such a manner that data related to a scan segment which data are detected at an end portion of a scan segment detection time interval are weighted stronger than data detected at a beginning portion of the scan segment detection time interval.

24. A method of examining an object of interest with a computer tomography apparatus, the method comprising the steps of:
rotating an electromagnetic radiation source and detecting elements around the object of interest;
emitting, by means of the electromagnetic radiation source, an electromagnetic radiation beam to the object of interest;
repeatedly detecting, by means of the detecting elements, scan segments of electromagnetic radiation emitted by the electromagnetic radiation source and passed through the object of interest;
repeatedly determining images of the object of interest based on an analysis of the detected scan segments so that the images are provided to be displayable essentially in real-time, wherein data related to a scan segment which data are detected at an end portion of a scan segment detection time interval are weighted stronger than data detected at a beginning portion of the scan segment detection time interval.

25. A computer-readable medium, in which a computer program of examining an object of interest with a computer tomography apparatus is stored which, when being executed by a processor, is adapted to control the steps of:
emitting, by means of the electromagnetic radiation source, an electromagnetic radiation beam to the object of interest;
repeatedly detecting, by means of the detecting elements, scan segments of electromagnetic radiation emitted by the electromagnetic radiation source and passed through the object of interest;
repeatedly determining images of the object of interest based on an analysis of the detected scan segments so that the images are provided to be displayable essentially in real-time, wherein data related to a scan segment which data are detected at an end portion of a scan segment detection time interval are weighted stronger than data detected at a beginning portion of the scan segment detection time interval.

26. A program element of examining an object of interest, which, when being executed by a processors, is adapted to control the steps of:
emitting, by means of the electromagnetic radiation source, an electromagnetic radiation beam to the object of interest;
repeatedly detecting, by means of the detecting elements, scan segments of electromagnetic radiation emitted by the electromagnetic radiation source and passed through the object of interest;
repeatedly determining images of the object of interest based on an analysis of the detected scan segments so that the images are provided to be displayable essentially in real-time, wherein data related to a scan segment which data are detected at an end portion of a scan segment detection time interval are weighted stronger than data detected at a beginning portion of the scan segment detection time interval.
